(19) United States
(12) Patent Application Publication
(10) Pub. No.: US 2014/0004196 A1
(43) Pub. Date: Jan. 2, 2014

(54) POLYAMIDE-AMINE DENDRIMER OR DERIVATIVE THEREOF-MATH1 GENE NANO PARTICLE AND USE THEREOF IN TREATMENT OF HEARING LOSS

(71) Applicants: Shi-Ming Yang, Beijing (CN); Nan Wu, Beijing (CN); Yan Wu, Beijing (CN); Dong Han, Beijing (CN); Wei-Wei Guo, Beijing (CN); Li-Dong Zhao, Beijing (CN); Suo-Quiang Zhai, Beijing (CN); Wei-Quiang Gao, Beijing (CN); Wei-Yen Young, Beijing (CN)

(72) Inventors: Shi-Ming Yang, Beijing (CN); Nan Wu, Beijing (CN); Yan Wu, Beijing (CN); Dong Han, Beijing (CN); Wei-Wei Guo, Beijing (CN); Li-Dong Zhao, Beijing (CN); Suo-Quiang Zhai, Beijing (CN); Wei-Quiang Gao, Beijing (CN); Wei-Yen Young, Beijing (CN)

(21) Appl. No.: 13/978,060

(22) PCT Filed: Jan. 4, 2012
(86) PCT No.: PCT/CN2012/070005

§ 371 Date: Sep. 16, 2013

(30) Foreign Application Priority Data
Jan. 4, 2011 (CN) 201110005066.6

(51) Int. Cl. A61K 48/00; C12N 15/85; A61K 38/17; A61K 9/14

(52) U.S. Cl. CPC A61K 48/0041; A61K 9/146; C12N 15/85; A61K 38/1709
USPC 424/489; 514/44 R; 435/320.1

(57) ABSTRACT

Polyamidoamine, its partially degraded products or its complexes-Math1 gene nanoparticles, method for preparing the same and use thereof, the gene nanoparticles can be produced through complex coacervating of polyamidoamine, or polyamidoamine complexes and a Math1 gene-containing plasmid. The gene nanoparticles are controllable in particle size, uniform in size, favorable for surface modification, can enhance the ability of expression and delivery of the Math1 gene, and is useful in a sensorineural hearing loss caused by hair cells loss due to noise, drug toxicity etc.

### CROSS REFERENCE TO RELATED APPLICATION

This application is a continuation of International application No. PCT/CN2012/070005, filed on Jan. 4, 2012 which claims the benefit of priority of CN application No, 201110005066.6 filed on Jan. 4, 2011, the content of which is incorporated herein by reference.

### FIELD OF THE INVENTION

The invention relates to the field of nanomaterial technology, in particular, to polyamidoamine, its partially degraded products or its complexes-Math1 gene nanoparticles, method for preparing said gene nanoparticles and their use in treating hearing loss.

### BACKGROUND

Recently, researches on gene therapy which takes non-viral materials as gene vectors have received wide attention, an important aspect among them is to use nanoparticles that are formed of cationic polymers and genes as the gene vectors to simulate structures similar to virus. Nanoparticle (NP) gene vector is a solid colloid nanoscale particle vector synthesized from polymer materials, which can wrap into or adsorb onto the surface of the nanoparticle the gene therapeutic molecules including DNA, RNA, PNA (peptide nucleic acid), dsRNA (double-stranded RNA) etc., and gradually release the gene therapeutic molecules through the degradation of the polymer materials after entrance of the nanoparticle to cells by cell endocytosis, so as to effect on gene therapy.

Numerous researches show that polycations can combine with genes in aqueous solution to form nanoparticles under certain conditions. Meanwhile, groups having special functions such as galactose, transferin can be introduced to the polycationic chain so as to allow the polycations genes nanoparticles to have functions similar to virus, for example, receptor regulation internalization, entry to nuclear and the like. Polycations as a gene vector is stable nanoparticles in the electrolyte environment in plasma, and will not inactivate during freeze drying storage.

Polyamidoamine (PAMAM) is a representative synthetic dendrimer, within physiological pH range, its amino groups on the surface carry a positive charge, and it is a common high molecular cationic polymer. PAMAM has a unique spherical shape, and a highly branched nanoscale dendritic structure. Its molecule is composed of three parts: core, inner repeated subunits and outer amino terminals, it has good hydrodynamic performance, easy to shape; meanwhile it has characters as low viscosity, high solubility, miscibility, high reactivity etc. In contrast, other cationic polymers such as chitosan and the like, need lower pH value for protonation, and has higher viscosity and poorer solubility. In terms of biological properties, PAMAM has excellent properties as non-immunity, low toxicity, and can be excreted through urine and feces.

As the generation of PAMAM increases, the amino groups on the terminal of the PAMAM dendrimers increase. The amino groups protonize under physiological pH, rendering the PAMAM to have a polycationic feature. The charges are liable to form stable complex with antibodies, nucleic acids and fluorescent groups by electrostatic interaction (Haensler and Szoka, 1993; Bielinska et al., 1996; Wang et al., 2000). Studies indicate that PAMAM can mediate the entry of nucleic acids, plasmids and the like to cells, and obtain the expression of target genes. Its mechanism is the PAMAM/DNA complexes having positive charges are liable to adhere to the negatively charged cell surfaces, to facilitate their entry to the interior of the cells and expression (Dennig J and Duncan E, 2002). The efficiency of transfection and cellular toxicity of PAMAM to cells will increase as its generation increase, such character is related to the target cells to be transfected.

Cochlea is the only organ for human to feel the outside acoustic stimuli, meanwhile it is a functionally specialized organ that is highly differentiated, its inner hair cells are mechanical-electrical sensors for feeling sound vibration, and plays an important role in hearing and balance. Any cause that makes degeneration, necrosis of the hair cells of the inner ear may cause dysfunction of hearing and balance. A traditional view believes that the cochlea hair cells of birds and mammals are differentiated in embryo phase and can not regenerate spontaneously, once the cochlea hair cells injure and lead to hearing loss, they can not restore naturally and have to be restored through artificial treatment, which has always been a worldwide difficulty. Researches in resent years show that the hair cells of the mammalian inner ear damaged from ototoxic drugs and noise can be regenerated through induction. Many grow factors such as TGF (transforming growth factor), fibroblast growth factors (FGF), epidermal growth factor (EGF), insulin-like growth factor (IGF) and the like, play important roles in the hair cells regeneration.

Math1 (Mammalian atonal homolog 1) is a basic helix-loop-helix (bHLH) gene, which is homologous gene to Drosophila Atoh1 gene in mice. Math1 gene is 1.18 kb in full length, contains one exton, its mRNA is 1065 bp in length, which encodes a protein composed of 354 amino acids, namely transcription factor Math1, the molecular weight of which is 38.2 kDa. Math1 gene is an essential gene for the differentiation and maturation of hair cells, and plays an important role in the regeneration of hair cells (Bermingham N A, Hassan B A, Price S D et al., Math1: an essential gene for the generation of inner ear hair cells. Science, 1999, 284:1837-1841).

Due to the limitation of viral vectors, the researches on the regeneration of inner ear hair cells conducted in animal bodies by viral vectors can not be applied to clinic. Nanoparticle as a novel gene vector, is advantageous for further researches and clinic applications of the hair cells regeneration owing to its non-immunogenicity, low toxicity, large loading capacity, easy preparation, structural stability, easy engineering and modification etc.

Currently, routes for introducing nanoparticle gene vectors into inner ear are mainly scala tympani perforation and round window membrane injection, introduction of the vectors to perilymphatic fluid through direct injection and micro-osmotic pump. Although these two manners are effective, they are invasive operation, damage cochlear scala tympani, have risks of inducing inflammation, perilymphorrhea, and healing injury. Since the round window membrane has the properties of semi-permeable membrane: absorption and secretion, and nanoparticle gene vectors have targeting and permeability, it is expected to realize the permeation through round window membrane as a novel technology.

However, so far there is no report on the preparation of PAMAM and its derivatives-Math1 gene nanoparticles and its in vitro transfection in cell or in vivo transfection in cochlear and an expression of Math1 gene.

### SUMMARY OF THE INVENTION

A purpose of the present invention is to provide a PAMAM-Math1 gene nanoparticle, for achieving a delivery of genes.

In an aspect of the invention, the PAMAM-Math1 gene nanoparticle comprises a PAMAM and a plasmid as show FIG. 4, which has a particle size of 100-200 nm, a distribution index of 0.10-0.25, zeta potential of about 10-50 mV, encapsulation efficiency of 90-95%. The PAMAM-Math1 gene nanoparticle is controllable in particle size, uniform in size, favorable for surface modification, and can enhance the ability of expression and delivery of the Math1 gene.

Another purpose of the present invention is to provide a partially degraded PAMAM products-Math1 gene nanoparticle, for achieving a delivery of genes.

In an aspect of the invention, the partially degraded PAMAM products-Math1 gene nanoparticle comprises partially degraded PAMAM products and a plasmid as shown in FIG. 4, which has a particle size of 100-200 nm, a distribution index of 0.10-0.25, zeta potential of about 10-50 mV, encapsulation efficiency of 90-95%. The partially degraded PAMAM products is obtained by thermal treatment of the PAMAM, particularly, the PAMAM is heated in aqueous solution at 5020 C.-100° C., and more particularly, heated for 2-48 hours.

Another purpose of the present invention is to provide a PAMAM complexes-Math1 gene nanoparticle, for achieving a delivery of genes.

In an aspect of the invention, the PAMAM complexes-Math1 gene nanoparticle comprises PAMAM complexes and a plasmid as shown in FIG. 4, which has a particle size of 100-200 nm, a distribution index of 0.10-0.25, zeta potential of about 10-50 mV, encapsulation efficiency of 90-95%. The PAMAM complexes are obtained by vibrating a PAMAM or its partially degraded products and a cyclodextrin in aqueous solutions. Particularly, the PAMAM or its partially degraded products are mixed with the cyclodextrin in a weight ratio of 1:10 to 10:1, more particularly, mixed and spinned for 10 s to 30 s. The cellular toxicity of the PAMAM complexes-Math1 gene nanoparticle is significantly lower.

Still another purpose of the present invention is to provide a method for preparing the PAMAM, partially degraded PAMAM products or its complexes-Math1 gene nanoparticles of the present application. The method is easy and simple, raw materials are easily available, no organic solvents and aldehyde agents are used as a crosslink agent, it has fast reaction, mild reaction condition, good repeatability, high stability, high practicability and wide applicability.

In an aspect of the invention, said PAMAM, partially degraded PAMAM products or its complexes-Math1 gene nanoparticles are prepared by coacervating of one of the above polymers and a Math1 gene-containing plasmid as shown in FIG. 4. Particularly, the PAMAM, partially degraded PAMAM products or its complexes are added to a Math1 gene-containing plasmid which is dissolved in PBS at room temperature, the Math1 gene-containing plasmid is wrapped by the polymers under an electrostatic interaction to form a nanoparticle suspension.

Specifically, the method comprises the following steps:

(1) Preparing an aqueous solution of PAMAM, its partially degraded products or its complexes of a concentration of 500-1500 μg/ml;

(2) Preparing a Math1 gene-containing plasmid in PBS solution of a concentration of 120-720 μg/ml;

(3) Mixing the solutions of steps (1), (2) in a N/P ratio (amino group of the PAMAM/phosphate group of the plasmid) of 30:1. to 1:10 to conduct complex coacervation reaction to obtain a suspension of PAMAM, partially degraded PAMAM products or its complexes-Math1 gene nanoparticles.

Wherein, a molecular weight of the PAMAM, partially degraded PAMAM products or PAMAM complexes is 500 Da-1,000,000 Da.

Wherein, a generation of the PAMAM is 1-10.

Wherein, the partially degraded PAMAM products are obtained by partial degradation (or breakage) through thermal treatment, which may further enhance a transfection level of genes in vitro.

Wherein, the PAMAM complexes are obtained through the combination of the PAMAM or partially degraded PAMAM products and the cyclodextrin, to reduce the cellular toxicity, in particular, is obtained in a weight ratio of 1:1.0 to 1.0:1 in aqueous solution by mixing and spinning.

Wherein, the Math1 gene-containing plasmid is shown in FIG. 4.

As compared to the prior art, the PAMAM, partially degraded PAMAM products or PAMAM complexes-Math1 nanoparticles prepared in the present invention at least have the following features:

(1) The selected dendritic molecules have advantages of stability, low viscosity, good solubility, non-immunogenicity, protonation under physiological pH ranges, high translocation efficiency to biological active substances.

(2) The prepared particles are adjustable in particle size and have a uniform size.

(3) The prepared particles have positive charges on the surface, and are favorable for surface modification.

(4) The prepared PAMAM, partially degraded PAMAM products or PAMAM complexes are combined with a Math1 gene-containing plasmid by electrostatic interaction to prepare nanoparticle, on one hand, to increase the stability of Math1 gene, allowing the Math1 gene to be eventually delivered to cells, on the other hand, to enhance its interaction with cell membrane and protect Math1 gene from being degraded by nuclease.

(5) The prepared partially degraded PAMAM products or PAMAM complexes-Math1 gene nanoparticles have higher transfection efficiency and lower cellular toxicity during cell transfection in vivo or in vitro.

(6) it is easy to realize a control on gene delivery by adjusting the proportion of respective components.

Still another purpose of the present invention is to provide use of PAMAM, partially degraded PAMAM products or PAMAM complexes-Math1 gene nanoparticles in the transfection of HEK 293 cells in vitro.

Still another purpose of the present invention is to provide use of PAMAM, partially degraded PAMAM products or PAMAM complexes-Math1 gene nanoparticles in the transfection of cochlea tissue ex vitro.

Still another purpose of the present invention is to provide use of PAMAM, partially degraded PAMAM products or PAMAM complexes-Math1 gene nanoparticles in the transfection of cochlea in vivo.

Still another purpose of the present invention is to provide use of PAMAM, partially degraded PAMAM products or PAMAM complexes-Math1 gene nanoparticles in hearing loss, the nanoparticles are useful in a sensorineural hearing loss caused by hair cells loss due to noise, drug toxicity etc.

### BRIEF DESCRIPTION OF THE DRAWINGS

FIG. 1 is a schematic view showing the formation of a PAMAM-Math1 nanoparticle;

FIG. 2 is a transmission electron micrograph showing a PAMAM complexes-Math1 nanoparticle;

FIG. 3 shows a particle distribution of a PAMAM complexes nanoparticle;

FIG. 4 shows a profile of plasmid PRK5-Math1-EGFP;

FIG. 5 shows a nucleic acid sequence of Math1 gene;

FIG. 6 shows an electrophosis profile, wherein lane 1: marker; 1: control group without transfection; 2: transfection group according to example 9; 3: transfection group according to example 10; 4: transfection group according to example 4;

FIG. 7 shows an expression of EGFP in 293T cells transfected by the PAMAM complexes-Math1 nanoparticle;

FIG. 8 shows the microinjection through post aurem round window membrane, a: anatomic mark of searching otic vesicle in post aurem, black arrow shows facial nerve, blue star shows sternocleidomastoid muscle; b: the sternocleidomastoid muscle is upward separated, post wail of otic vesicle (blue arrow) and posterior belly of digastric muscle (blue star) are visible; c: posterior belly of digastric muscle (blue star) is backward separated to expose the posterior upper bone wall of otic vesicle (direction indicated by black arrow); d: removal the rear bone wall over the posterior upper to the otic vesicle, and facial nerve to the otic vesicle; 3: the otic vesicle is opened and round window niche (black arrow), stapedial artery (blue arrow) are visible; f: microinjection through punctuation of round window membrane (black arrow indicates the needle); and

FIG. 9 shows an inner ear tissue having Math1-EGFP protein expressed; 1: inner hair cell region; 2: column cell region; 3: outer hair cell region.

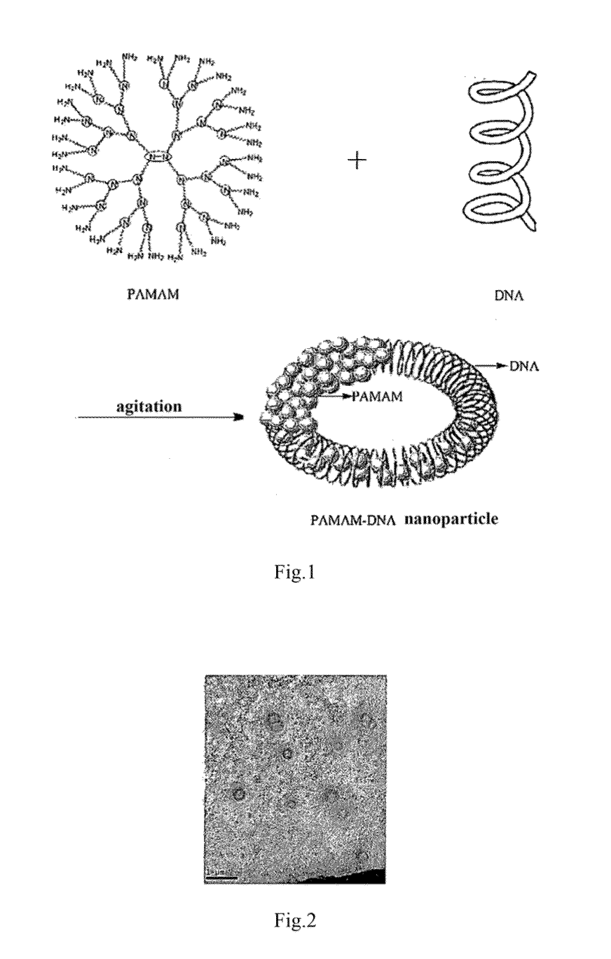

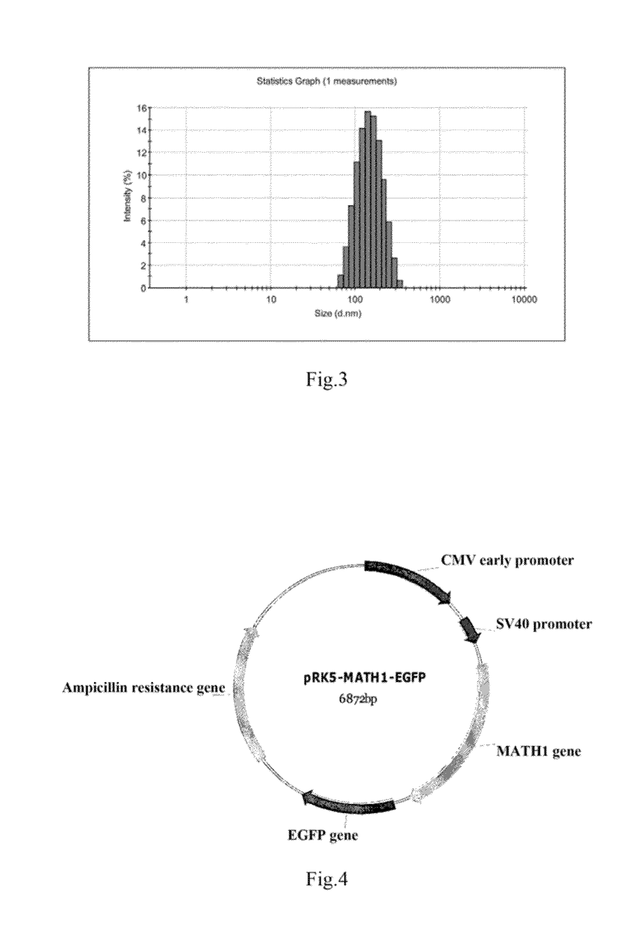

### DETAILED DESCRIPTION

Hereinafter, description to the present invention will be made in details in reference to the examples.

Unless otherwise indicated, reagents, medicines, and materials in the following examples are commercially available, methods used in the examples can be referred to Molecular Cloning A Laboratory Manual (Sambrook and Russell, 2001).

### EXAMPLE 1

### Construction of PRK5-Math1 Plasmid

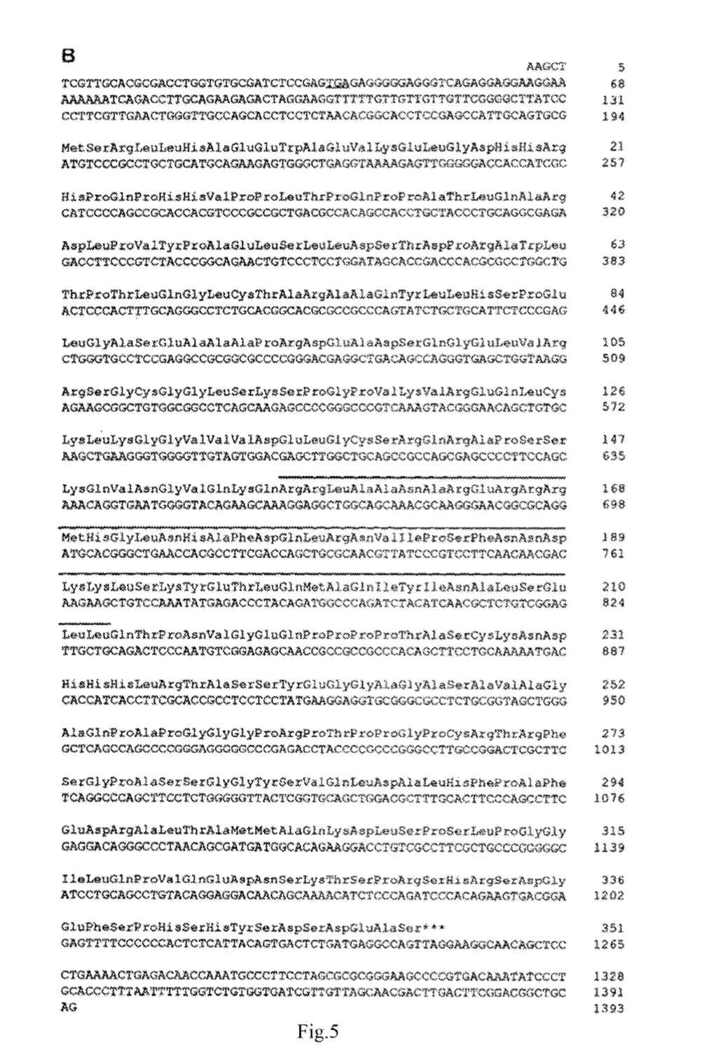

16-Days brain tissue from embryonic mice was extracted for total RNA by Trizol method, cDNA was synthesized by reverse transcription, Math1 gene containing F-box was synthesized by PCR method, and ECOR1 and BamH1 enzyme restriction cites were added at 5′ and 3′ ends thereof. The PCR amplified product was digested by ECOR1 and BamH1, purified and ligated to a PRK5 plasmid (Clontech) which was also digested by ECOR1 and BamH1, to construct PRK5-Math1 plasmid. Wherein Math1 gene has a sequence shown in FIG. 5.

Primers for Amplification:

F: 5′-GGAATTAAAATAGTTGGGGGACC-3′;
R: 5′-TGGACAGCTTCTTGTTGGCTT-3′.

Condition for Amplification: 94° C. 5 min; 94° C. 1 min; 58° C. 40 sec; 72° C. 40 sec; 35 cycles, extension at 72° C. 5 min.

### EXAMPLE 2

### Construction of PRK5-Math1 EGFP Plasmid

Plasmid pEGFP-C2 (invitrogen) containing EGFP gene and the PRK5-Math1 plasmid of Example 1 were double digested by Hpa1 and XbaI 1 enzyme, respectively, purified and recovered, and ligated by T4 ligase to construct PR K5-Math1-EGFP plasmid.

### EXAMPLE 3

### Amplification and Purification of PRK5-Math1 EGFP

100 μl competent E. coli DH 5a bacteria was added to 5 μl PRK5-Math1-EGFP plasmid, homogenized, ice bathed for 30 min, heat shocked at 42° C. for 1 min, ice bathed for 2 min, 800 μl LB medium was added and cultured at 37° C. for 1 hour. 100 μl broth was coated on a plate containing ampicillin and inverted cultured at 37° C. for 16 hours. Single colonies were picked from the plate, inoculated in 5 ml LB liquid medium containing ampicillin, shaken at constant temperature of 37° C. overnight, allowing the bacteria to grow to post-log phase. The plasmid was extracted in accordance with the instruction of plasmid extraction kit (QIAGEN).

5 U endonuclease (not more than 1/10 of the total reaction volume) was added to 0.5˜1 μg plasmid, the reaction volume was 20 μl, bathed for 2 h at proper temperature, and a small amount of samples were taken for agarose gel electrophoresis to detect the digestion result.

### EXAMPLE 4

100 μl of 500 μg/ml PAMAM solution was added to 100 μl of 720 μg/ml PRK5-Math1-EGFP plasmid in PBS solution, mixed immediately at vortex mixer for 30 sec, and proceeded to incubation at room temperature for 0.5 hour to obtain a nano suspension of PAMAM-PRK5-Math1-EGFP plasmid. Dynamic Light Scattering showed its particular size was 118.6 nm, distribution index was 0.187; and zeta potential was 42±1.17 (mV) as determined by Zeta Potential Analyzer.

### EXAMPLE 5

100 μl of 500 μg/ml PAMAM solution which is partially degraded by thermal treatment at 50° C. for 24 h was added to 100 μl of 720 μg/ml PRK5-Math1-EGFP plasmid in PBS solution, mixed immediately at vortex mixer for 30 sec, and continued to incubate at room temperature for 0.5 hour to obtain a nano suspension of PAMAM-PRK5-Math1-EGFP plasmid. Dynamic Light Scattering determines its particular size was 105.1 nm, distribution index was 0.206; zeta potential was 39±1.12 (mV) as determined by Zeta Potential Analyzer.

### EXAMPLE 6

100 μl of 500 μg/ml PAMAM solution which is partially degraded by thermal treatment at 50° C. for 24 h was added to 50 μl of 720 g/ml PRK5-Math1-EGFP plasmid in PBS solution, mixed immediately at vortex mixer for 30 sec, and continued to incubate at room temperature for 0.5 hour to obtain a nano suspension of PAMAM-PRK5-Math1-EGFP plasmid. Dynamic Light Scattering determines its particular size was 104.2 nm, distribution index was 0.198; zeta potential was 41.6±1.19 (mV) as determined by Zeta Potential Analyzer.

### EXAMPLE 7

100 μI of 500 μg/ml PAMAM solution which is partially degraded by thermal treatment at 100° C. for 24 h was added to 25 μl of 720 μg/ml PRK5-Math1-EGFP plasmid in PBS solution, mixed immediately at vortex mixer for 30 sec, and continued to incubate at room temperature for 0.5 hour to obtain a nano suspension of PAMAM-PRK5-Math1-EGFP plasmid. Dynamic Light Scattering determines its particular size was 102.9 nm, distribution index was 0.202; zeta potential was 42.9±1.23 (mV) as determined by Zeta Potential Analyzer.

### EXAMPLE 8

100 μl of 500 μg/ml PAMAM solution which is partially degraded by thermal treatment at 50° C. for 48 h was added to 100 μl of 720 μg/ml PRK5-Math1-EGFP plasmid in PBS solution, mixed immediately at vortex mixer for 30 sec, and continued to incubate at room temperature for 0.5 hour to obtain a nano suspension of PAMAM-PRK5-Math1-EGFP plasmid. Dynamic Light Scattering determines its particular size was 101.5 nm, distribution index was 0.211; zeta potential was 37±1.28 (mV) as determined by Zeta Potential Analyzer.

### EXAMPLE 9

Beta-cyclodextrin was added to PAMAM solution in a mass ratio of 1:10, mixed for 10 s. Then 100 μl of 500 μg/ml PAMAM complexes was added to 100 μl of 720 μg/ml PRK5-Math1-EGFP plasmid in PBS solution, mixed immediately at vortex mixer for 30 sec, and continued to incubate at room temperature for 0.5 hour to obtain a nano suspension of PAMAM complexes-PRK5-Math1-EGFP plasmid. Dynamic Light Scattering determines its particular size was 129.2 nm, distribution index was 0.245; zeta potential was 35±1.31 (mV) as determined by Zeta Potential Analyzer.

### EXAMPLE 10

Beta-cyclodextrin was added to PAMAM solution which is partially degraded by thermal treatment at 50° C. for 24 h in a mass ratio of 10:1, mixed for 10 s. Then 100 μl of 500 μg/ml PAMAM complexes was added to 100 μl of 720 μg/ml PRK5-Math1-EGFP plasmid in PBS solution, mixed immediately at vortex mixer for 30 sec, and continued to incubate at room temperature for 0.5 hour to obtain a nano suspension of PAMAM complexes-PRK5-Math1-EGFP plasmid. Dynamic Light Scattering determines its particular size was 130.2 nm, distribution index was 0.247; zeta potential was 39±1.19 (my) as determined by Zeta Potential Analyzer.

### EXAMPLE 11

### In Vitro Transfection of PAMAM Complexes-PRK5-Math1-EGFP Nanoparticles in HEK 293 Cells and Expression of Math1 Protein

HEK 293T cells were seeded to a 35 mm Petri dish one day before transfection, until the cells reached 80% confluence for transfection. During transfection, cells were washed twice using DMEM medium containing 10% FBS, and 2 ml of preheated DMEM medium containing 10% FBS was added to each dish. The nano suspension prepared according to the above examples were gently shaken for sufficient mixing (the concentration of PAMAM complexes nanoparticle was 4 ng/μl), and 300 μl nano solution was added to each dish, the dishes were gently shaken for sufficient mixing, cultured at 5% CO₂ incubator for 24-48 hours.

293T cells were collected after cultured at 36.5° C.±0.5° C. for 48 hours (about 10⁷ cells), and extracted for RNA by Trizol method. RNA was resolved by adding 30 μl DEPC water, 2 μl was taken for measuring RNA content by UV spectrophotometer, and RNA was frozen storage at −80° C.

PCR Amplification

Template denaturation: RNA prepared in the above steps were heated at 65° C. for 5min, to melt the secondary structure, and then cooled immediately on ice.

Reaction system for template denatureation:

| Components | Volume |
|---|---|
| RNA | 5.0 μl |
| Oligo dT (20) | 1.0 μl |
| dNTPs | 2.0 μl |
| Template | 3.0 μl (200 ng) |
| ddH₂O | 4.0 μl |

Reaction system for reverse transcription:

| Components | Volume |
|---|---|
| 5x Buffer | 5.0 μl |
| 0.1M DTT | 1.0 μl |
| RNAseOUT ™ | 1.0 μl |
| Reverse transcriptase III | 3.0 μl |
| ddH₂O | 1.0 μl |

The following reaction was carried out after sufficient mixing: 50° C. for 60 min, 7° C. for 5 min, 1 μl of RNase H was added and reacted at 37° C. for 20 min. The thus obtained reverse transcripted product was used as template for the following PCR amplification reaction, or froze at −20° C.

Reaction system for PCR amplification:

| Components | Volume |
|---|---|
| 10x Buffer (—Mg²⁺) | 5.0 μl |
| 50 mM MgCl₂ | 1.5 μl |
| 10 mM dNTP | 1.0 μl |
| 10 μM Primer 1 | 1.0 μl |
| 10 μM Primer 2 | 1.0 μl |
| Tag enzyme | 0.4 μl |
| cDNA | 2.0 μl |
| DEPC H₂O | 1.0 μl |

PCR reaction procedure: pre-denaturation for 5 min at 95° C. and then enter cycle of denaturation for 45 sec at 95° C., renaturation for 45 sec at 58° C., extension for 1min at 72° C., totally 40 cycles, and then extension for 5 min at 72° C., the obtained PCR product was subjected to the follow reaction or frozen at −20° C.

RT-PCR reaction products were determined by 1% agarose gel.

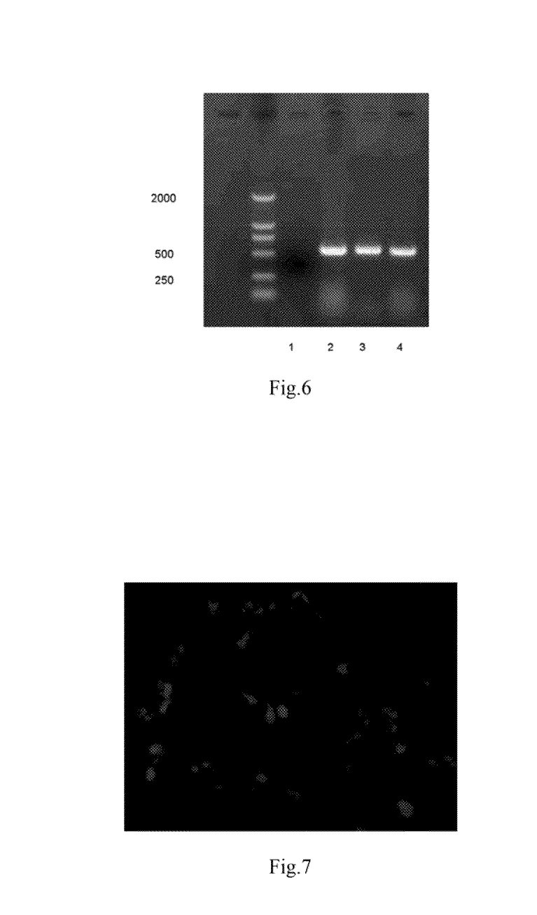

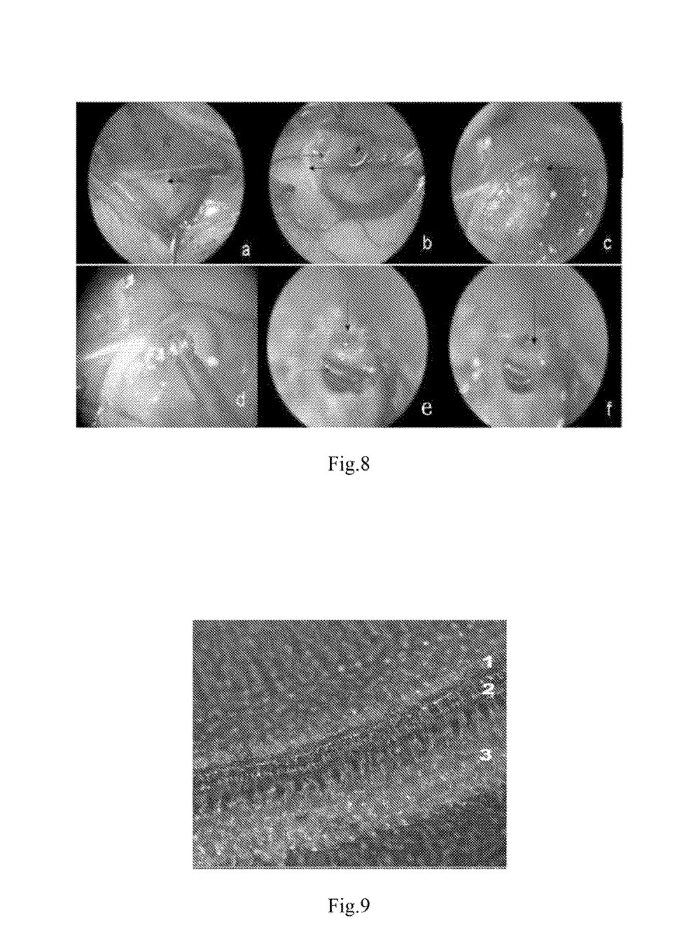

As shown in FIG. 6, Math1 gene can be translated in HFK 293 cells, to product Math1 protein.

Further, as shown in FIG. 7, 293T cells transfected by PAMAM complexes-PRK5-Math1-EGFP nanoparticles can express Math1-EGFP gene, indicating that the PAMAM complexes-PRK5-Math1-EGFP nanoparticles can deliver target genes to living cells and expression.

### EXAMPLE 12

### In Vitro Transfection of PAMAM Complexes-Math1 Gene Nanoparticle in Cochlea tissue of SD rats

The SD rats were immersed in alcohol 3 days after birth, decollated and removed for otic vesicle; the removed cochlea tissue was rapidly placed in Hank's buffer at 4° C.; the cochlea tissue was separated to remove the spiral ligament and stria vascularis; the basement membrane was divided into three sections of base layer, middle layer and top layer; DMEM containing 10% FBS was added into 24-well culture plate; the basement membrane tissue was plated on the culture plate carefully, and placed in 5% CO₂ incubator at 37° C.; the medium was changed every other day.

Six days after culture when the basement membrane tissue was well adherent, it was washed twice with DMEM containing 10% FBS for transfection, 3 ml of DMEM containing 10% FBS which was preheated at 37° C. was added to each dish; the PAMAM complexes nano solution prepared in the examples was mixed gently and sufficiently (the concentration of PAMAM complexes nanoparticle was 4 ng/μl), and 300 μl nano solution was added to each dish and gently shaken for sufficient mixing, cultured at 5% CO₂ incubator for 24-48 hours to observe the results.

### EXAMPLE 13

### In Vivo Transfection of PAMAM Complexes-Math1 Gene Nanoparticle in Cochlea Tissue of SD Rats through Punctuation Fiber Injection of Round Window Membrance

3-week-old healthy SD rats of 120 g-130 g in weight, male or female, sensitive in auricle reflex, normal in eardrum of both ears and not infected, were taken. The rats were anesthetized using chloral hydrate for animals (Beijing) per 4.5 ml/kg weight, and were preserved in isothermic bags at 37° C. After anesthesia, the otic vesicle of the right ear was exposed by the ventral route under strict aseptic condition, the otic vesicle was opened with an electric drill under operational microscope, to expose the cochlea, and punched at the basal scala tympani with the puncture needle to outflow the perilymph. The optimum concentration of in vivo transfection was established according to the optimum experimental conditions for in vitro cell transfection level, with the diluent being artificial perilymph; a solution of 5 μl PAMAM complexes-PRK5-Math1-EGFP gene nanoparticle was slowly injected (about 5 min) therein through scala tympani perforation, then a small piece of muscle was filled to block the perforation of the otic vesicle, the wounds were layered-sutured. Administration was conducted via scala tympani perforation fibers injection, the administrative method is simple and easy, reliable in transfection, efficient and relative small harassment on inner ear.

### EXAMPLE 14

### Immunohistochemistry of Inner Ear Tissue of SD Rats Transfected with of PAMAM Complexes-PRK5-Math1-EGFP Gene Nanoparticle

7 days after transfected by the PAMAM complexes-PRK5-Math1-EGFP gene nanoparticle, the rats were decollated, then rapidly removed for otic vesicle, fixed with 4% paraformaldehyde for 1 h, and subjected to cochlea basement membrane stretch; observed by confocal fluorescence microscope after stained by immunohistochemistry, with excitation light of 488 nm; the inner and outer hair cells having Math1-EGFP expressed is green fluorescence. As shown in FIG. 9, inner and outer hair cells express Math1-EGFP, showing green fluorescence. Accordingly, the PAMAM complexes-PRK5-Math1-EGFP gene nanoparticle can effectively transfect different cells including inner and outer hair cells of the inner ear and expression, promote the regeneration of hair cells and can be used for the treatment of sensorineural hearing loss.

It is noted that although the embodiments of the present invention are described in details above, the embodiments are only exemplary, those skilled in the art are able to combine the ranges of various parameters disclosed herein based on the teach from the above embodiments to obtain various technical solutions. Further, those skilled in the art are also able to make modifications and changes to the present invention without departing from the spirit of the present invention, which is within the scope of the present invention.)

What is claimed is:

1. A polyamidoamine-Math1 gene nanoparticle, comprising a polyamidoamine and a plasmid as shown in FIG. 4, with a particle size of 100-200 nm, a distribution index of 0.10-0.25, zeta potential of about 10-50 mV, encapsulation efficiency of 90-95%.

2. A partially degraded polyamidoamine products Math1 gene nanoparticle, comprising a polyamidoamine and a plasmid as shown in FIG. 4, with a particle size of 100-200 nm, a distribution index of 0.10-0.25, zeta potential of about 10-50 mV, encapsulation efficiency of 90-95%, the partially degraded polyamidoamine products are obtained by thermal treatment of an intact polyamidoamine molecule.

3. A polyamidoamine complexes-Math1 gene nanoparticle, comprising polyamidoamine complexes and a plasmid as shown in FIG. 4, with a particle size of 100-200 nm, a distribution index of 0.10-0.25, zeta potential of about 10-50 mV, encapsulation efficiency of 90-95%, the complexes are obtained by mixing a polyamidoamine or its partially degraded products and a cyclodextrin, the partially degraded products is obtained by thermal treatment of an intact polyamidoamine molecule.

4. A method for preparing nanoparticles, wherein a suspension of nanoparticles is obtained by subjecting an aqueous solution of polyamidoamine, partially degraded products of polyamidoamine or polyamidoamine complexes and a Math1 gene-containing plasmid in PBS solution to complex coacervation, said partially degraded products are obtained by thermal treatment of an intact polyamidoamine molecule, said complexes are obtained by mixing the polyamidoamine or its partially degraded products with a cyclodextrin, said Math1 gene-containing plasmid is shown in FIG. 4.

5. The method of claim 4, characterized in that, comprising:
(1) preparing the aqueous solution of polyamidoamine or its partially degraded products or the aqueous solution of polyamidoamine complexes of a concentration of 500-1500 μg/ml;
(2) preparing Math1 gene-containing plasmid in PBS solution of a concentration of 120-720 μg/ml;
(3) mixing the solutions of step (1), (2) in a ratio of amino group of the polyamidoamine/phosphate group of the plasmid of 30:1 to 1:10 to conduct complex coacervation reaction to obtain a suspension of polyamidoamine-Math1 gene nanoparticles, a suspension of polyamidoamine partially degraded products-Math1 gene nanoparticles, or a suspension of polyamidoamine complexes-Math1 gene nanoparticles.

6. The method of claim 4, characterized in that, said polyamidoamine has a molecule weight of 500 Da-1,000,000 Da.

7. The method of claim 4, of characterized in that, said thermal treatment is conducted in aqueous solution at 50-100° C. for 2-48 hours, said polyamidoamine or its partially degraded products is mixed with the cyclodextrin in a weight ratio of 1:10 to 10:1.

8. A nanoparticle prepared by the method of claim 4, characterized in that, said nanoparticle has a particle size of 100-200 nm, a distribution index of 0.10-0.25, zeta potential of about 10-50 mV, encapsulation efficiency of 90-95%.

9. Use of the nanoparticles according to claim 1, for in vitro transfection of HEK 231T cells, ex vitro transfection of cochlea tissue or in vivo transfection of cochlea.

10. The method of claim 5, characterized in that, said polyamidoamine has a molecule weight of 500 Da-1,000,000 Da.

11. The method of claim 5, characterized in that, said thermal treatment is conducted in aqueous solution at 50-100° C. for 2-48 hours, said polyamidoamine or its partially degraded products is mixed with the cyclodextrin in a weight ration of 1:10 to 10:1.

12. Use of the nanoparticle according to claim 2 for in vitro transfection of HEK 293T cells, ex vitro transfection of cochlea tissue or in vivo transfection of cochlea.

13. Us of the nanoparticles according to claim 3, for in vitro transfection of HEK 293T cells, ex vitro transfection of cochlea tissue or in vivo transfection of cochlea.

14. Use of the nanoparticles according to claim 8 for in vitro transfection of HEK 293T cells, ex vitro transfection of cochlea tissue or in vivo transfection of cochlea.


SEQUENCE LISTING (2 sequences)

SEQ ID NO: 1 - DNA, 1396 bp, Mus musculus
aagcttcgttgcacgcgacctggtgtgcgatctccgagtgagagggggagggtcagaggaggaaggaaaaaaaatcagaccttgcagaagagactaggaaggtttttgttgttgttgttgttcggggcttatccccttcgttgaactgggttgccagcacctcctctaacacggcacctccgagccattgcagtgcgatgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaagagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagttaggaaggcaacagctccctgaaaactgagacaaccaaatgcccttcctagcgcgcgggaagccccgtgacaaatatccctgcaccctttaatttttggtctgtggtgatcgttgttagcaacgacttgacttcggacggctgcag

SEQ ID NO: 2 - protein, 351 aa, Mus musculus
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS